(19) United States
(12) Patent Application Publication
(10) Pub. No.: US 2006/0159714 A1
(43) Pub. Date: Jul. 20, 2006

(54) Use of an antioxidant in a dermatological and/or cosmetic composition

(76) Inventor: Jean-Noel Thorel, Paris (FR)

Correspondence Address: OLIFF & BERRIDGE, PLC, P.O. BOX 19928, ALEXANDRIA, VA, 22320, US

(21) Appl. No.: 10/562,443

(22) PCT Filed: Jul. 7, 2004
(86) PCT No.: PCT/FR04/01768

§ 371 Date: Jan. 18, 2006

(30) Foreign Application Priority Data
Jul. 7, 2003 (FR) 03/08288

(51) Int. Cl. A61K 8/73; A61K 8/49; A61K 8/37

(52) U.S. Cl. 424/401; 424/70.13

(57) ABSTRACT

The invention relates to a dermatological and/or cosmetic composition which is intended for the treatment of acne, seborrheic dermatitis and skin disorders associated with the formation of comedones. The invention is characterised in that it contains: as active principle, at least one lipophilic antioxidant which is selected from the group comprising gallates, flavonoids, butyl-hydroxytoluene (BHT), butyl-hydroxyanisole (BHA), octadecenedioic acid and hydroxydecanoic acid; and at least one second active principle in the form of a hydrophilic antioxidant selected from the group comprising mannitol, vitamin C, lysine azelate, rutin and quercetin.

The present invention relates to the treatment of acne and skin disorders related to the formation of comedos.

The comedo is the primary lesion of acne vulgaris and this lesion results from the obstruction by cells of the wall of the follicle of the canal, preventing the sebum produced by sebocytes (cells of the sebaceous glands) from reaching the surface of the skin. The mixture of sebum and of cells forms a plug, referred to as comedo, and brings about, in the pore thus plugged, the proliferation of bacteria which normally live on the skin, such as Propionibacterium acnes and granulosum, and yeast, in particular Malassezia furfur. These bacteria have the distinguishing feature of metabolizing the triglycerides of the sebum with the release of the fatty acids which bring about the inflammation of the tissues.

The seborrheic dermatitis resulting therefrom is reflected by an eruption of red plaques or blotches, covered with yellowish greasy scales, more or less pruriginous, predominant in the areas rich in sebaceous glands. On the face, the topography of the lesions is suggestive: groove between the nose and the lips, root of the eyebrows, scalp, wings of the nose, folds of the auriculae, conchae of the ears, external auditory canals. On the scalp, frequent attack is reflected by a more or less seborrheic dandruff condition. On the trunk, two frequent areas are noticed in man: the sternum and the region between the two shoulder blades.

The sebum is a product of fatty secretion which is rich in fatty acids and in particular in squalene, an aliphatic hydrocarbon comprising 30 carbon atoms which is the precursor of cholesterol. The sebum plays an important positive role, in particular in the protection of the skin, but it has also been established, since 1969 (Cunliffe, W. J. et al., Lancet, I, 685, 1969, The Pathogenesis of Acne), that there exists a correlation between the level of secretion of sebum and the severity of the acne.

Thus, in modern cosmetics and dermatology, much research has been carried out on developing compositions in order to reduce and control the excessive secretions of the sebaceous glands, in particular in order to reduce the unsightly consequences, such as the greasy and oily appearance of the skin and scalp, but also in order to reduce the formation of comedos and the resulting inflammation.

These compositions have not insignificant side effects, such as dry skin, feelings of tightness, indeed even inflammation, due to the removal of the sebum, which can no longer play its protective role.

As regards the treatments for the hair, mention will be made of the hair compositions disclosed in FR 2 099 582 which include, in solution, at least one phenolic derivative, said compositions being intended to deodorize the hair but which, in addition to this deodorizing action, slow down the secretions of sebum, and the application of which appears to have the result either of a strong slow down in the secretions or of their modification in the direction of a lower fluidity.

Recent scientific studies have shown that one of the constituents of the sebum, squalene and in particular the oxidation products of squalene, have comedogenic and also irritant properties (Saint-Léger et al., British J. of Dermatology, 114, 543-552, 1986, Chiba K. et al., The J. of Toxicological Sciences, 25, 77-83, 2000, and Uchino, T. et al., Biol. Pharm. Bulletin, 25(5), 605-610, 2002).

More specifically, it has been established that the composition of the sebum of patients affected by acne is highly enriched in squalene.

The Applicant Company has also shown that the oxidation of the lipids constituting the sebum results in an increase in the viscosity of said sebum and an increase in the formation of comedos, it no longer being possible for the less fluid sebum to flow out of the follicular canals, this oxidation being potentially brought about by the bacterial components and ultra-violet radiation, resulting in the formation of viscous polymers which thicken the sebum and which are highly comedogenic.

The present invention thus relates to compositions which improve the quality of the sebum by preventing it from thickening and thus reduce the induction of keratinocyte proliferation, resulting in a decrease in comedogenesis.

Surprisingly, the results with regard to skin suffering from acne are obtained without modifying the flow of sebum, that is to say without affecting the amount of sebum produced, but by reducing its comedogenicity, by correcting its quality and by preventing it from thickening.

The invention relates more specifically to compositions which make it possible to maintain the fluidity of the sebum and more particularly to a composition for dermatological and/or cosmetic use intended for the treatment of acne, seborrheic dermatitis and skin disorders related to the formation of comedos, characterized in that it comprises, as active principle, at least one lipophilic antioxidant chosen from the group consisting of gallates, flavonoids, butylated hydroxytoluene (BHT), butylated hydroxy-anisole (BHA), octadecenedioic acid and hydroxydecanoic acid.

It also relates to a composition as defined above, characterized in that it additionally comprises at least one second active principle, a hydrophilic antioxidant chosen from the group consisting of mannitol, vitamin C, lysine azelate, rutin and quercetin.

The invention also relates to a composition in which the lipophilic antioxidant is dodecyl gallate.

The invention also relates to a composition in which the lipophilic antioxidant is propyl gallate.

The invention also relates to a composition in which the lipophilic antioxidant is octyl gallate.

The flavonoids can, for example, be contributed by plant extracts, such as Ginkgo biloba or green tea extracts.

The amount of active principle, that is to say of hydrophilic or lipophilic antioxidant or the total amount of the two antioxidants, is between 0.0001 and 20% by weight of the composition and preferably between 0.0001 and 10% by weight of the composition.

It is preferably between 0.001 and 2% by weight of the composition.

The invention also relates to a process for rendering the sebum fluid, characterized in that it comprises the application, to the skin or scalp, of a composition according to the invention as defined above.

This composition comprises, in a pharmacologically acceptable medium, at least one active principle, that is to say one lipophilic antioxidant.

The pharmacologically acceptable medium, that is to say the formulation environment, must be neither oxidizable nor oxidizing, that is to say that the excipients used must be nonoxidizable and stable under the conditions of application to the skin and must not bring about oxidation.

The invention thus relates to a composition as defined above, characterized in that it does not comprise excipients capable of oxidizing under the conditions of use.

The compositions according to the invention can optionally comprise various nonoxidizable additives, such as suspending agents, emulsifiers, anionic, cationic, nonionic or amphoteric polymers, proteins, vitamins, surfactants, mineral or vegetable oils, silicone waxes, gums and/or resins, thickening agents, acidifying or basifying agents, solvents, pH stabilizers, UV stabilizers, preservatives, antibacterials and antifungals, fragrances or other adjuvants commonly used in cosmetics or in dermatology.

Preferably, the compositions according to the present invention are provided in a form suitable for administration by the topical skin route and cover all cosmetic or dermatological forms. These compositions comprise a cosmetically and/or dermatologically acceptable medium, that is to say a medium compatible with the skin or hair, including head hair. These compositions can in particular be in the form of creams, O/W, W/O or multiple emulsions, solutions, suspensions, gels, milks, lotions, sticks or powders suitable for application to the skin, lips and/or hair.

The invention also relates to the use of at least one liphophilic antioxidant in the preparation of a dermatological and/or cosmetic composition for the treatment of acne and/or of skin disorders due to the formation of comedos.

It relates more particularly to the use as defined above, characterized in that the lipophilic antioxidant is chosen from the group consisting of gallates and flavonoids.

In an alternative form, the lipophilic antioxidant is chosen from the group consisting of butylated hydroxytoluene (BHT), butylated hydroxyanisole (BHA), octadecenedioic acid and hydroxydecanoic acid.

The invention also relates to said use when the lipophilic antioxidant is dodecyl gallate.

The invention also relates to said use when the lipophilic antioxidant is propyl gallate.

The invention also relates to said use when the lipophilic antioxidant is octyl gallate.

The flavonoids can, for example, be contributed by plant extracts, such as Ginkgo biloba or green tea extracts.

The invention also relates to the use of a hydrophilic antioxidant in combination with a lipophilic antioxidant in the preparation of a dermatological and/or cosmetic composition for the treatment of acne and/or of skin disorders due to the formation of comedos.

It also relates to a use as defined above, characterized in that the hydrophilic antioxidant is chosen from the group consisting of mannitol and vitamin C.

In an alternative form, the hydrophilic antioxidant is chosen from the group consisting of lysine azelate, rutin and quercetin.

According to the invention, the amount of hydrophilic or lipophilic antioxidant or the total amount of the two antioxidants used is between 0.0001 and 20% by weight of the composition and preferably between 0.0001 and 10% by weight of the composition.

It is preferably between 0.001 and 2% by weight of the composition.

Other advantages and characteristics of the invention will become more clearly apparent on reading the examples, which are given by way of examples and without implied limitation.

### EXAMPLE 1

### Preparation of an Active Mixture

Sepigel 305 (Polyacrylamide and C13-14

Isoparaffin and Laureth-7) 2.00%

Dodecyl gallate 0.0001%

Mannitol 0.50%

Preservative (parabens) 0.20%

EDTA (sequestering agent) 0.10%

Water q.s. for 100%

### EXAMPLE 2

### Effect of a Composition According to the Invention

A composition according to the invention comprising, as active principle, dodecyl gallate, mannitol and a Ginkgo biloba extract was tested on a group of approximately thirty volunteers.

The subjects, on average 23 years (18 to 34 years) old, exhibit polymorphic juvenile acne of moderate intensity, that is to say exhibiting approximately 40 retentional or inflammatory lesions on average.

Only the retentional lesions, that is to say microcysts and comedos, are observed, by counting over the entire face, except the nasal pyramid.

The composition according to the invention was applied to the face twice daily for 8 weeks.

After applying for 8 weeks, a decrease in the retentional lesions is recorded for at least 40% of the subjects, with 20% of the subjects exhibiting a decrease of 50 to 90% in the microcysts, 15% a decrease of 50 to 90% in the comedos and 10% complete disappearance of the comedos.

### EXAMPLE 3

### Preparation of Compositions

These compositions were obtained by simple mixing of the various components. The amounts shown are given as percentage by weight.

Oil-in-water emulsion

| | |
|---|---|
| Montanov 68 (Cetearyl Alcohol and | 5.00% |
| Cetearyl Glucoside) | |
| Jojoba oil | 5.00% |
| BHT | 0.05% |
| Isopropyl palmitate | 7.00% |
| Glycerol | 5.00% |
| Allantoin | 0.10% |
| Mannitol | 3.00% |
| Sepigel 305 (Polyacrylamide and C13-14 | 0.30% |
| Isoparaffin and Laureth-7) | |
| Phenonip | 0.50% |
| Fragrance | 0.50% |
| Water | q.s. for 100% |

Gel

| | |
|---|---|
| Carbopol Ultrez 10 (sol. a 2%) | 25.00% |
| Triethanolamine | 0.50% |
| Mannitol | 2.00% |
| Dodecyl gallate | 0.0001% |
| Preservative | 0.20% |
| EDTA (sequestering agent) | 0.10% |
| Fragrance | 0.50% |
| Water | q.s. for 100% |

Lotion

| | |
|---|---|
| Monopropylene glycol | 1.00% |
| Allantoin | 0.30% |
| Glycerol | 1.00% |
| Cetiol HE (PEG-7 Glyceryl Cocoate) | 1.00% |
| Lysine azelate | 5.00% |
| BHA | 0.01% |
| Preservative | 0.20% |
| Fragrance | 0.50% |
| Water | q.s. for 100% |

Foaming gel for seborrheic greasy skin

| | |
|---|---|
| Compound (Mannitol 90% and Ascorbyl Palmitate 10%) | 2.00% |
| Fragrance | 0.30% |
| Sodium chloride | 1.00% |
| 57% Glycolic acid in water | 0.50% |
| Copolymer of oxyethylenated (60 EO) | 0.90% |
| hydrogenated tallow alcohol and of myristyl glycol | |
| Glycerol | 3.00% |
| 38% N-Disodium N-carboxyethoxyethyl-N- | 5.00% |
| (cocoylamidoethyl)aminoacetate in water | |
| 28% Sodium lauryl ether sulfate (C12-14 70/30) | 14.30% |
| (2.2 EO) in water | |
| Coconut fatty acid diethanolamide | 0.70% |
| Mixture of oxyethylenated (26 EO)/ | 1.00% |
| oxypropylenated (26 PO) butyl alcohol, | |
| oxyethylenated (40 EO) hydrogenated | |
| castor oil in water | |
| Demineralized water | q.s. for 100% |

Treating gel for seborrheic skin

| | |
|---|---|
| Ascorbyl palmitate | 1.00% |
| Fragrance | 0.20% |
| Xanthan gum | 1.00% |
| Glycerol | 2.00% |
| Ethyl alcohol | 20.00% |
| Rutin | 0.10% |
| Mixture of oxyethylenated (26 EO)/ | 1.00% |
| oxypropylenated (26 PO) butyl alcohol, | |
| oxyethylenated (40 EO) hydrogenated | |
| castor oil in water | |
| Demineralized water | q.s. for 100% |

Purifying lotion for skin suffering from
acne and for dandruff conditions

| | |
|---|---|
| BHA | 0.05% |
| Propyl gallate | 0.0005% |
| Fragrance | 0.20% |
| Ethyl alcohol | 20.00% |
| Glycerol | 2.00% |
| Mixture of oxyethylenated (26 EO)/ | 1.00% |
| oxypropylenated (26 PO) butyl alcohol, | |
| oxyethylenated (40 EO) hydrogenated | |
| castor oil in water | |
| Octopirox | 0.20% |
| Demineralized water | q.s. for 100% |

Other active principles can be combined with the antioxidants in order to supplement the action of the compositions according to the invention and to reinforce the effectiveness and the tolerance thereof. This results in compositions for which the various components of the action make it possible to meet the complex requirements of skin with a tendency toward acne.

The compositions according to the invention can additionally comprise active principles having a keratolytic activity chosen from esters of α-hydroxy acids and/or salicylic acid which make it possible to eliminate and prevent the formulation of clusters of corneocytes which can also promote the formation of comedos.

They can also additionally comprise a zinc salt, for example zinc gluconate, having a seboregulatory action, by its inhibitory action on 5α-reductase, and, at high concentration, a bactericidal action on Propionibacterium acnes, the proliferation of which in the comedo is characteristic of acne.

The addition to the compositions according to the invention of high concentrations of an active principle comprising zinc makes it possible to supplement the action of antibiotic treatments, such as treatments with erythromycin, in local applications or systemically.

The compositions according to the invention can also comprise an anti-inflammatory or soothing active principle, such as 18β-glycyrrhetic acid (enoxolone), the endogenous anti-inflammatory role of which would be due to the inhibition of the enzyme responsible for the conversion of cortisol to cortisone, or a Ginkgo biloba extract described as inhibitor of the inflammatory cascade.

The action of these active principles is supplemented by a formulation base comprising glycerol and xylitol, chosen for their moisturizing properties.

1. A composition for dermatological and/or cosmetic use intended for the treatment of acne, seborrheic dermatitis and skin disorders related to the formation of comedos, characterized in that it comprises, as active principle, at least one lipophilic antioxidant chosen from the group consisting of gallates, flavonoids, butylated hydroxytoluene (BHT), butylated hydroxyanisole (BHA), octadecenedioic acid and hydroxydecanoic acid and at least one second active principle, a hydrophilic antioxidant chosen from the group consisting of mannitol, vitamin C, lysine azelate, rutin and quercetin.

2. The composition as claimed in claim 1, characterized in that the lipophilic antioxidant is dodecyl gallate.

3. The composition as claimed in claim 1, characterized in that the antioxidant is propyl gallate.

4. The composition as claimed in claim 1, characterized in that the antioxidant is octyl gallate.

5. The composition as claimed in claim 1, characterized in that the amount of active principle is between 0.0001 and 20% by weight of the composition and preferably between 0.0001 and 10% by weight of the composition.

6. The composition as claimed in claim 1, characterized in that the amount of active principle is between 0.001 and 2% by weight of the composition.

7. The composition as claimed in claim 1, characterized in that it does not comprise excipients capable of oxidizing under the conditions of use.

8. The use of a lipophilic antioxidant in the preparation of a dermatological and/or cosmetic composition for the treatment of acne and/or of skin disorders due to the formation of comedos.

9. The use of a hydrophilic antioxidant in combination with a lipophilic antioxidant in the preparation of a dermatological and/or cosmetic composition for the treatment of acne and/or of skin disorders due to the formation of comedos.

10. The use as claimed in claim 9, characterized in that the lipophilic antioxidant is chosen from the group consisting of gallates and flavonoids.

11. The use as claimed in claim 10, characterized in that the hydrophilic antioxidant is chosen from the group consisting of mannitol and vitamin C.

12. A process for rendering the sebum fluid, characterized in that it comprises the application, to the skin or scalp, of a composition as claimed in claim 1.
